(19) United States
(12) Patent Application Publication
(10) Pub. No.: US 2025/0043229 A1
(43) Pub. Date: Feb. 6, 2025

(54) Device for Detecting Solution State

(71) Applicant: LG Chem, Ltd., Seoul (KR)

(72) Inventors: Youbin Kwon, Daejeon (KR); Jaegil Choi, Daejeon (KR); Youngsoo Song, Daejeon (KR); Ye Hoon Im, Daejeon (KR); Jaehyun Kim, Daejeon (KR); Ikjin Choi, Daejeon (KR); Gibok Nam, Daejeon (KR); Gyeongho Seon, Daejeon (KR)

(73) Assignee: LG Chem, Ltd., Seoul (KR)

(21) Appl. No.: 18/718,150

(22) PCT Filed: Jul. 6, 2023
(86) PCT No.: PCT/KR2023/009556

§ 371 Date: Jun. 10, 2024

(30) Foreign Application Priority Data
Aug. 16, 2022 (KR) 10-2022-0102208
Jun. 30, 2023 (KR) 10-2023-0085322

(51) Int. Cl. C12M 1/34; C12M 1/00; C12M 3/00

(52) U.S. Cl. CPC C12M 41/00; C12M 23/46; C12M 29/04

(57) ABSTRACT

A device for detecting a solution state includes: a probe including a detecting portion having at least one open surface to measure the solution state; a mesh disposed on a path through which a solution is introduced to the detecting portion; and a support portion disposed on the outside of the probe, and having an opening surrounding the at least one open surface of the detecting portion. The opening defines the path through which the solution is introduced to the detecting portion, and the mesh is mounted in the opening of the support portion.

### CROSS-REFERENCE TO RELATED APPLICATION

The present application is a national phase entry under 35 U.S.C. § 371 of International Application No. PCT/KR2023/009556 filed Jul. 6, 2023, which claims priority from Korean Patent Application No. 10-2022-0102208 filed in the Korean Intellectual Property Office on Aug. 16, 2022 and Korean Patent Application No. 10-2023-0085322 filed in the Korean Intellectual Property Office on Jun. 30, 2023, the entire contents of which are incorporated herein by reference.

### TECHNICAL FIELD

The present disclosure relates to a device for detecting a solution state, and more particularly, to a device for detecting a solution state which may include a mesh disposed in a path through which a solution is introduced into a probe for detecting the solution state so as to prevent a bubble from entering a detecting portion of the probe, thereby preventing an error from occurring in the detected solution state.

### BACKGROUND ART

3-Hydropropionic Acid (3-HP) may be a platform compound that may be converted into many other useful chemicals, including acrylic acid, and thus, may be widely applied to the production of pigments, paints, fibers, or the like. 3-HP may be a representative biodegradable material that may be produced by fermenting colon bacillus.

The colon bacillus may be cultured and fermented in a culture fluid in a reactor. A spectrometer may be installed in this reactor to detect a concentration of the colon bacillus in the culture fluid in real time. In addition, various detection devices may be installed in the reactor to detect a state (e.g., concentration, capacitance, or inductance) of the culture fluid, thereby predicting a state of the colon bacillus. These detection devices may be typically configured to detect the state of the culture fluid flowing into the detecting portion in real time.

An agitating device may also be disposed in the reactor for cultivating the colon bacillus. The agitating device may be configured to agitate the culture fluid and supply oxygen to the colon bacillus in the culture fluid. When the agitating device is operated and agitates the culture fluid, a bubble or the like may occur in the culture fluid, and the occurring bubble may be introduced into the detecting portion of the detection device along with the culture fluid. As such, when the bubble is present in, or passes through, the detecting portion, the state of the culture fluid detected by the detection device may not be uniform. That is, an error may occur in the state of the culture fluid detected by the detection device.

The above information disclosed in this section is provided only to assist in the further understanding of the background of the present disclosure, and may thus include information not included in the prior art already known to those skilled in the art to which the present disclosure pertains.

### Technical Problem

The present disclosure attempts to provide a device for detecting a solution state which may include a mesh disposed in a path through which a solution is introduced into a probe for detecting the solution state so as to prevent a bubble from entering the probe.

### Technical Solution

According to an embodiment, provided is a device for detecting a solution state, the device including: a probe including a detecting portion having at least one open surface to detect the solution state; a mesh disposed on a path through which a solution is introduced into the detecting portion; and a support portion disposed on an outside of the probe, and having an opening surrounding the at least one open surface of the detecting portion, wherein the opening defines the path through which the solution is introduced into the detecting portion, and the mesh is mounted in the opening of the support portion.

An inner surface of the support portion may be spaced apart from an outer surface of the probe by a predetermined distance.

The support portion may be detachably mounted on an outer surface of the probe through a fastening portion.

The support portion may include first and second support portions spaced apart from each other by a predetermined distance, and the first and second support portions may be detachably assembled to each other for the mesh to be mounted at the predetermined distance between the first and second support portions.

The first and second support portions may be screwed to each other.

The second support portion may be inserted into the first support portion while being spaced apart from the first support portion by the predetermined distance, and a first thread portion may be formed on an inner surface of a lower portion of the first support portion, a second thread portion may be formed on an outer surface of a lower portion of the second support portion, and the first thread portion and the second thread portion may be screwed to each other.

Upper ends of the first and second support portions may be open.

A lower end of the first support portion may be open, and a lower end of the second support portion may be open, closed, or openable.

An inner surface of the second support portion and an outer surface of the probe may be spaced apart from each other by the predetermined distance.

A fastening portion may be formed in an upper portion of the first support portion, and the support portion may be detachably mounted on an outer surface of the probe through the fastening portion.

The fastening portion may be disposed above an upper end of the second support portion.

### Advantageous Effects

According to the present disclosure, the mesh may be disposed in the path through which the solution is introduced into the probe so as to prevent the bubble from entering the probe.

In addition, the mesh may be mounted on the support portion, and the support portion may be detachably mounted on the probe. Accordingly, the mesh and the support portion may thus be easily installed or replaced.

In addition, the support portion may be made larger than the probe by the predetermined size, thereby preventing the bubble from escaping out of the support portion and causing the error in the detection value of the state even if the bubble is present between the support portion and the probe.

Other effects which may be acquired or predicted by the embodiments of the present disclosure are disclosed directly or implicitly in the detailed description of the embodiments of the present disclosure. That is, various effects predicted based on the embodiments of the present disclosure are disclosed in the detailed description described below.

### DESCRIPTION OF THE DRAWINGS

Embodiments in the specification may be better understood by reference to the following description in connection with the accompanying drawings in which like reference numerals refer to identical or functionally similar elements.

FIG. 1 is a perspective view of a device for detecting a solution state according to an embodiment of the present disclosure.

FIG. 2 is a perspective view of a probe according to an embodiment of the present disclosure.

FIG. 3 is a perspective view of a support portion according to an embodiment of the present disclosure.

FIG. 4 is a perspective view of a first support portion included in the support portion of FIG. 3.

FIG. 5 is a perspective view of a second support portion included in the support portion of FIG. 3.

FIG. 6 shows a spectrum depending on the presence or absence of a bubble in the probe.

FIG. 7 shows an example of a spectrum of a culture fluid detected using a device for detecting a solution state according to an embodiment of the present disclosure.

FIG. 8 shows an example of a spectrum of a culture fluid detected using a device for detecting a solution state according to the prior art.

It should be understood that the drawings referenced above are not necessarily drawn to scale, and present a rather simplified representation of various preferred features showing the basic principles of the present disclosure. For example, specific design features of the present disclosure, including its specific dimension, orientation, position and shape, are determined in part by the particular intended application and environment of use.

### DETAILED DESCRIPTION

A term used herein is only to describe a specific embodiment, and is not intended to limit the present disclosure. A term of a singular number used herein is intended to include its plural number unless the context clearly indicates otherwise. It should also be understood that the terms “include” and/or “including,” when used in the specification, specify the presence of the recited features, integers, steps, operations, elements and/or components, and do not exclude the presence, or addition of, one or more of other features, integers, steps, operations, elements, components and/or groups thereof. A term “and/or” used herein includes any one or all combinations of the associated listed items.

A device for detecting a solution state according to an embodiment of the present disclosure may include a mesh disposed in a path through which a solution is introduced into a probe so as to prevent a bubble from entering the probe. Accordingly, it is possible to accurately detect the solution state. In addition, the mesh may be mounted on a support portion, and the support portion may be detachably mounted on the probe. Accordingly, the mesh and the support portion may thus be easily installed or replaced. In addition, the support portion may be made larger than the probe by a predetermined size, thereby preventing the bubble from escaping out of the support portion and causing an error in a detection value of the state even if the bubble is present between the support portion and the probe.

Hereinafter, an embodiment of the present disclosure is described in detail with reference to the accompanying drawings.

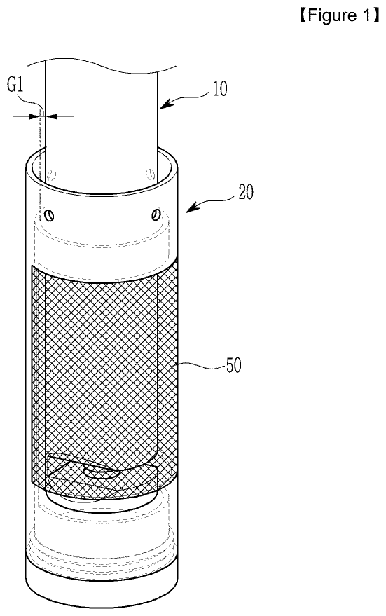

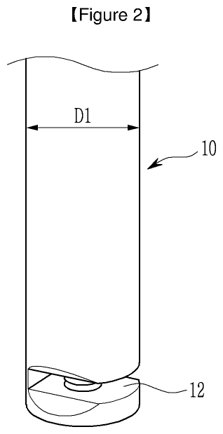

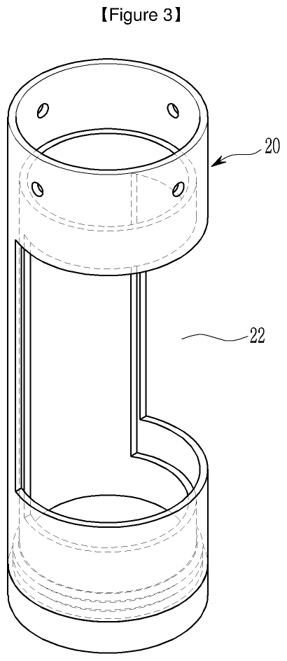

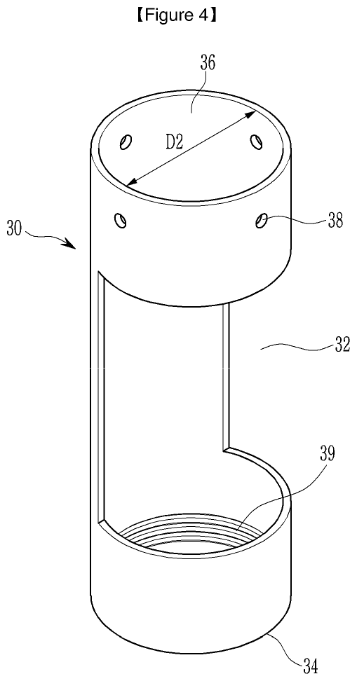

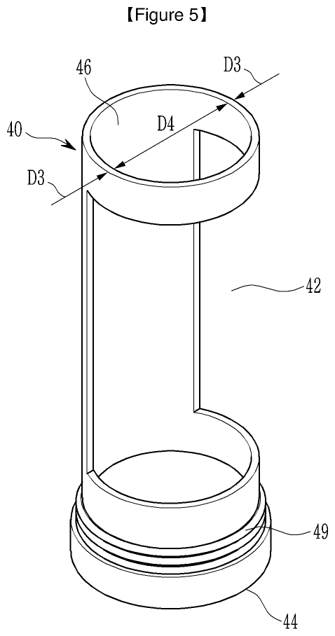

FIG. 1 is a perspective view of a device for detecting a solution state according to an embodiment of the present disclosure; FIG. 2 is a perspective view of a probe according to an embodiment of the present disclosure; FIG. 3 is a perspective view of a support portion according to an embodiment of the present disclosure; FIG. 4 is a perspective view of a first support portion included in the support portion of FIG. 3; and FIG. 5 is a perspective view of a second support portion included in the support portion of FIG. 3.

As shown in FIGS. 1 to 5, a device for detecting a solution state according to an embodiment of the present disclosure may include a probe 10, a support portion 20, and a mesh 50.

As shown in FIGS. 1 and 2, the probe 10 may be configured to detect the solution state (e.g., a concentration, a capacitance, or an inductance of a solution). The probe 10 may have a pillar shape, and have an upper portion fixed to, or passing through, an upper portion or a lid of a reactor, and a lower portion immersed in the solution in the reactor. For example, a detecting portion 12 may be disposed in the lower portion of the probe 10, and may be configured to detect the state of the solution which is present in, or passes through, the detecting portion 12. For this purpose, the lower portion of the probe 10, especially at least the detecting portion 12, may be disposed to be immersed in the solution. In addition, at least one surface of the detecting portion 12 may be open and function as an inlet through which the solution is introduced into the detecting portion 12 or an outlet through which the solution escapes from the detecting portion 12. In this specification, it is exemplified that the probe 10 has a circular column shape and the support portion 20 has a cylinder shape. However, the respective shapes of the probe 10 and the support portion 20 are not limited to the circular column shape and the cylinder shape. When the probe 10 has the circular column shape, the probe 10 may be shown as having a diameter of D1.

As shown in FIGS. 1 and 3, the support portion 20 may be provided to mount the mesh 50 around the detecting portion 12, and detachably mounted on the probe 10. When the probe 10 has the circular column shape, the support portion 20 may have a hollow cylinder shape and surround the probe 10. The support portion 20 may have an opening 22 formed therein. The opening 22 may surround a part of an outer peripheral surface of the probe 10, for example, at least the open surface of the detecting portion 12. Therefore, the solution in the reactor may be introduced into the detecting portion 12 only through the opening 22. That is, the opening 22 may form a path through which the solution is introduced into the detecting portion 12 of the probe 10.

The support portion 20 may be provided as a single item, or may be provided as two or more items and may be detachably assembled to each other. In an example, the support portion 20 may include a first support portion 30 and a second support portion 40. Here, the description exemplifies that the number of items included in the support portion 20 is two. However, it should be understood that the number of items included in the support portion 20 is not necessarily limited to two.

As shown in FIGS. 1, 3 and 4, the first support portion 30 may correspond to an outer shell of the support portion 20. The first support portion 30 may include a first opening 32 corresponding to the opening 22. In addition, the first support portion 30 includes a lower end 34 and an upper end 36. The lower end 34 of the first support portion 30 may be open so that the second support portion 40 may be inserted into the first support portion 30 from bottom to top. Similarly, the upper end 36 of the first support portion 30 may be open so that the probe 10 may be inserted into the first support portion 30 from top to bottom, that is, so that the first support portion 30 may be moved from bottom to top to a mounting position of the first support portion 30 on the probe 10. When the first support portion 30 has the hollow cylinder shape, the first support portion 30 may be shown as having an inner diameter of D2.

The first support portion 30 may be detachably assembled to the second support portion 40. In an example, a first thread portion 39 may be formed on a lower portion of an inner peripheral surface of the first support portion 30, and may be screwed to the second support portion 40. In addition, the first support portion 30 may be detachably mounted on the outer peripheral surface of the probe 10. For this purpose, a fastening portion 38 may be formed in the upper portion of the first support portion 30. In an example, the fastening portion 38 may be a fastening hole. In this case, a corresponding fastening hole may be formed in the outer peripheral surface of the probe 10. Accordingly, the first support portion 30 may be mounted on the outer peripheral surface of the probe by inserting a screw, a pin, or the like into the fastening hole of the first support portion 30 and the corresponding fastening hole of the probe 10. In addition, the first support portion 30 may be detached from the probe 10 by removing the screw, the pin, or the like from the fastening hole and/or the corresponding fastening hole. Here, the description exemplifies that the fastening portion 38 is the fastening hole, but the present disclosure is not limited thereto.

As shown in FIGS. 1, 3 and 5, the second support portion 40 may correspond to an inner shell of the support portion 20. The second support portion 40 may include a second opening 42 corresponding to the opening 22. Therefore, when the first support portion 30 and the second support portion 40 are assembled to each other, the first opening 32 and the second opening 42 are coupled to each other to thus form the opening 22.

The second support portion 40 may include a lower end 44 and an upper end 46. The lower end 44 of the second support portion 40 may be open, closed, or openable by a lid, or the like. In general, the bubble occurring by an agitating device may have a low specific gravity to float on top of the solution. Therefore, the bubble may not be introduced to the detecting portion 12 through the open lower end 44 of the second support portion 40. Therefore, even when the lower end 44 of the second support portion 40 is open, the open lower end may have little effect on an operation of the detecting portion 12. The upper end 46 of the second support portion 40 may be open so that the probe 10 may be inserted into the second support portion 40 from top to bottom, that is, so that the second support portion 40 may be moved from bottom to top to a mounting position of the second support portion 40 on the probe 10. When the second support portion 40 has the hollow cylinder shape, the second support portion 40 may be shown as having an outer diameter of D3 and the second support portion 40 may be shown as having an inner diameter of D4.

As described above, the second support portion 40 may be detachably assembled to the first support portion 30. In an example, a second thread portion 49 may be formed on a lower portion of an outer peripheral surface of the second support portion 40, and may be screwed to the first thread portion 39 of the first support portion 30. In addition, the upper end 46 of the second support portion 40 may be disposed below the fastening portion 38 so that the support portion 20 formed by coupling the first support portion 30 with the second support portion 40, may be detachably mounted on the outer peripheral surface of the probe 10. That is, the fastening portion 38 may be disposed above the upper end 46 of the second support portion 40. Therefore, the support portion 20 may be detachably mounted on the outer peripheral surface of the probe 10 by using only the fastening portion 38 without forming a fastening portion corresponding to the fastening portion 38 in the second support portion 40.

In an example, the inner diameter D2 or the inner surface of the first support portion 30 may be spaced apart from the outer diameter D3 or the outer surface of the second support portion 40 by a predetermined distance. The predetermined distance may be provided to install the mesh 50 between the inner diameter D2 or the inner surface of the first support portion 30 and the outer diameter D3 or the outer surface of the second support portion 40. The predetermined distance is not limited thereto, and may range from 0.3 mm to 1 mm. In addition, the outer diameter D1 or the outer surface of the probe 10 and the inner diameter D4 or the inner surface of the second support portion 40 may be spaced apart from each other by the predetermined distance G1. Even if the bubble is present or introduced between the probe 10 and the support portion 20, the bubble may escape through the predetermined distance G1. The predetermined distance G1 is not limited thereto, and may range from 2 mm to 5 mm.

The mesh 50 may be mounted in the opening 22 surrounding the open surface of the detecting portion 12 of the probe 10. That is, the mesh 50 may be disposed on the path through which the solution is introduced into the detecting portion 12 of the probe 10, thus preventing the bubble in the solution from being introduced into the detecting portion 12 of the probe 10. As described above, the mesh 50 may be mounted at the predetermined distance between the inner diameter D2 or the inner surface of the first support portion 30 and the outer diameter D3 or the outer surface of the second support portion 40. In addition, the lower end of the mesh 50 may not deviate from the predetermined distance by being supported by the first and second thread portions 39 and 49 screwed to each other. That is, the mesh 50 may deviate only upward from the predetermined distance, which means that the mesh 50 does not deviate from the support portion 20 without a user's intention to detach the mesh 50. The mesh 50 is not limited to any particular size as long as the mesh has a large size enough to prevent the bubble from being introduced into the detecting portion 12. In an example, the mesh 50 could be a 300 mesh or a 500 mesh. In another example, the mesh 50 may have a size determined based on the size, flow rate, or the like of the bubble occurring in an operation condition of the reactor. For example, the mesh 50 may be 100 mesh to 1000 mesh.

A material of the mesh 50 is not particularly limited, and the mesh 50 may be used as long as the mesh 50 is made of a material that does not corrode in a liquid. In an example, the material of the mesh 50 may be a metal material such as a steel use stainless (SUS) material or a plastic material, which has a strong corrosion resistance.

In addition, a hole in the mesh 50 is not limited to any particular shape as long as the mesh 50 may prevent passage of the bubble and reduce its flow rate. In an example, the hole may have various shapes, such as a circle or a polygon.

Hereinafter, the description briefly describes a process of installing the device for detecting a solution state on the probe 10. In an example, the probe 10 may have the circular column shape, and may be configured to include the upper portion fixed to or passing through the upper portion or lid of the reactor and the lower portion immersed in the solution in the reactor.

First, the support portion 20 may be formed by screwing the first and second thread portions 39 and 49 to each other and assembling the first and second support portions 30 and 40 to each other. Here, the opening 22 may be formed by coupling the first and second openings 32 and 42 to each other. In addition, the upper end 46 of the second support portion 40 may be disposed below the fastening portion 38, and the inner diameter D2 of the first support portion 30 and the outer diameter D3 of the second support portion 40 may be spaced apart from each other by the predetermined distance.

In this state, the mesh 50 may be inserted downward into the predetermined distance between the inner diameter D2 of the first support portion 30 and the outer diameter D3 of the second support portion 40. The mesh 50 may be mounted in the opening 22, which is the path through which the solution is introduced into the detecting portion 12 of the probe 10.

The support portion 20 equipped with the mesh 50 may then be mounted on the outer peripheral surface of the probe 10 through the fastening portion 38. Here, the mesh 50 may surround the open surface of the detecting portion 12, and the outer diameter D1 of the probe 10 and the inner diameter D4 of the second support portion 40 may be spaced apart from each other by the predetermined distance G1, thus forming a space for the bubble to escape between the probe 10 and the support portion 20.

Hereinafter, the description describes an effect of the device for detecting a solution state according to an embodiment of the present disclosure.

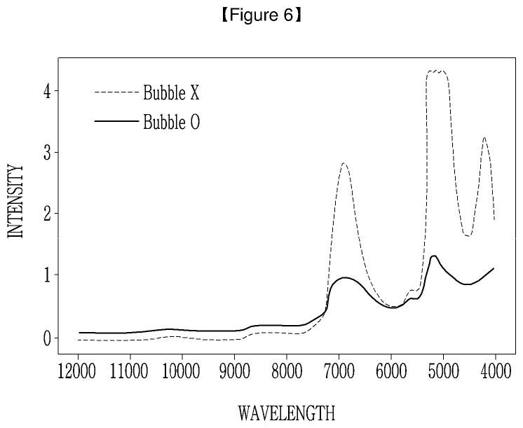

FIG. 6 shows a spectrum depending on the presence or absence of the bubble in the probe. FIG. 6 shows the spectrum for the cases where the bubble is present and absent in the detecting portion 12 of the probe 10 when the reactor is stopped. In FIG. 6, a dotted line represents the spectrum when the bubble is absent in the detecting portion 12, and a solid line represents the spectrum when bubble is present in the detecting portion 12.

As may be seen from the dotted line in FIG. 6, when the bubble is absent in the detecting portion 12, a peak having an intensity of 7000 Å, 5000 Å, or 4000 Å may be generated. However, as may be seen from the solid line in FIG. 6, when the bubble is present in the detecting portion 12, the bubble may cause the error in the detection value of the detecting portion 12, and the peak may not be generated or may have a low intensity even when the peak is generated.

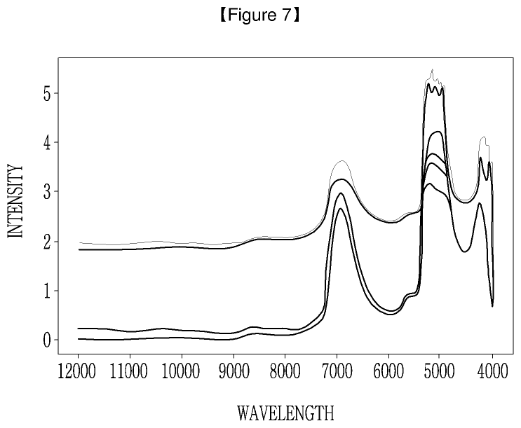

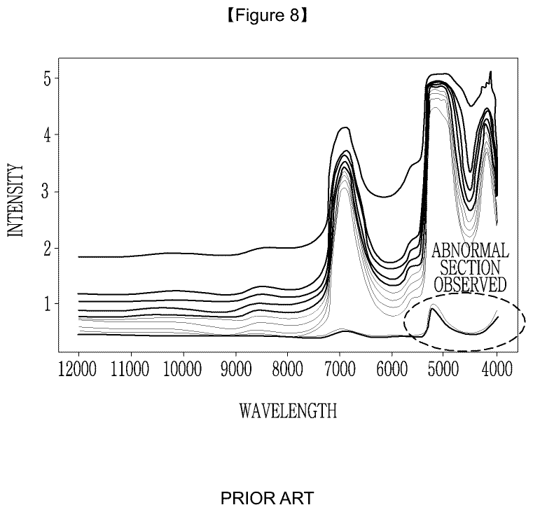

FIG. 7 shows an example of a spectrum of the culture fluid detected using the device for detecting a solution state according to an embodiment of the present disclosure; and FIG. 8 shows an example of a spectrum of a culture fluid detected using a device for detecting a solution state according to the prior art. FIGS. 7 and 8 show a change in the spectrum that is detected in a process of cultivating the colon bacillus for 24 hours.

As shown in FIG. 7, it may be seen that the spectrum indicated by the dotted line in FIG. 6 is generated when the spectrum of the culture fluid is detected using the device for detecting a solution state according to an embodiment of the present disclosure. That is, the device for detecting a solution state according to an embodiment of the present disclosure may prevent the bubble from being introduced to the detecting portion 12, thus acquiring an accurate spectrum.

As shown in FIG. 8, an abnormal section may be observed at a low wavelength when detecting the spectrum of the culture fluid using the device for detecting a solution state according to the prior art. That is, the device for detecting a solution state according to the prior art may not have a means to prevent the bubble from being introduced to the detecting portion, and an error may occur in the detection value.

Although the embodiments of the present disclosure have been described hereinabove, the scope of the present disclosure is not limited thereto, and all equivalent modifications easily modified by those skilled in the art to which the present disclosure pertains are intended to fall within the scope and spirit of the present disclosure.

1. A device for detecting a solution state, comprising:
a probe including a detecting portion having at least one open surface so as to detect the solution state;
a mesh disposed on a path through which a solution is introduced into the detecting portion; and
a support portion disposed on an outside of the probe, and having an opening surrounding the at least one open surface of the detecting portion,
wherein the opening defines the path through which the solution is introduced into the detecting portion, and
wherein the mesh is mounted in the opening of the support portion.

2. The device of claim 1, wherein
an inner surface of the support portion is spaced apart from an outer surface of the probe by a predetermined distance.

3. The device of claim 1, wherein
the support portion is detachably mounted on an outer surface of the probe through a fastening portion.

4. The device of claim 1, wherein:
the support portion includes first and second support portions spaced apart from each other by a predetermined distance, and
the first and second support portions are detachably assembled to each other so as to allow the mesh to be mounted at the predetermined distance between the first and second support portions.

5. The device of claim 4, wherein
the first and second support portions are screwed to each other.

6. The device of claim 5, wherein:
the second support portion is inserted into the first support portion while being spaced apart from the first support portion by the predetermined distance, and
a first thread portion is formed on an inner surface of a lower portion of the first support portion, a second thread portion is formed on an outer surface of a lower portion of the second support portion, and the first thread portion and the second thread portion are screwed to each other.

7. The device of claim 4, wherein
upper ends of the first and second support portions are open.

8. The device of claim 4, wherein:
a lower end of the first support portion is open, and
a lower end of the second support portion is open, closed, or openable.

9. The device of claim 4, wherein
an inner surface of the second support portion and an outer surface of the probe are spaced apart from each other by a predetermined distance.

10. The device of claim 4, wherein:
a fastening portion is formed in an upper portion of the first support portion, and
the support portion is detachably mounted on an outer surface of the probe through the fastening portion.

11. The device of claim 10, wherein
the fastening portion is disposed above an upper end of the second support portion.
